SEQUENCE LISTING (16 sequences)

SEQ ID NO: 1 - protein, 8 aa, Artificial Sequence
  Description of Artificial Sequence Synthetic Peptide
GPQGIAGQ

SEQ ID NO: 2 - protein, 8 aa, Artificial Sequence
  Description of Artificial Sequence Synthetic Peptide
VPMSMRGG

SEQ ID NO: 3 - protein, 8 aa, Artificial Sequence
  Description of Artificial Sequence Synthetic Peptide
IPVSLRSG

SEQ ID NO: 4 - protein, 8 aa, Artificial Sequence
  Description of Artificial Sequence Synthetic Peptide
RPFSMIMG

SEQ ID NO: 5 - protein, 8 aa, Artificial Sequence
  Description of Artificial Sequence Synthetic Peptide
VPLSLTMG

SEQ ID NO: 6 - protein, 8 aa, Artificial Sequence
  Description of Artificial Sequence Synthetic Peptide
VPLSLYSG

SEQ ID NO: 7 - protein, 8 aa, Artificial Sequence
  Description of Artificial Sequence Synthetic Peptide
IPESLRAG

SEQ ID NO: 8 - protein, 5 aa, Artificial Sequence
  Description of Artificial Sequence Synthetic Peptide
PAPRG

SEQ ID NO: 9 - protein, 5 aa, Artificial Sequence
  Description of Artificial Sequence Synthetic Peptide
LDPRS

SEQ ID NO: 10 - protein, 6 aa, Artificial Sequence
  Description of Artificial Sequence Synthetic Peptide
LVPRGS

SEQ ID NO: 11 - protein, 8 aa, Artificial Sequence
  Description of Artificial Sequence Synthetic Peptide
AQCRKYCP

SEQ ID NO: 12 - protein, 8 aa, Artificial Sequence
  Description of Artificial Sequence Synthetic Peptide
TKPKMLPP

SEQ ID NO: 13 - protein, 6 aa, Artificial Sequence
  Description of Artificial Sequence Synthetic Peptide
KPVSDF

SEQ ID NO: 14 - protein, 6 aa, Artificial Sequence
  Description of Artificial Sequence Synthetic Peptide
HHHHHH

SEQ ID NO: 15 - protein, 9 aa, Artificial Sequence
  Description of Artificial Sequence Synthetic Peptide
CGPQGIAGQ

SEQ ID NO: 16 - protein, 14 aa, Artificial Sequence
  Description of Artificial Sequence Synthetic Peptide
HHHHHHGPQGIAGQ

(19) United States
(12) Patent Application Publication
(10) Pub. No.: US 2011/0165244 A1
(43) Pub. Date: Jul. 7, 2011

(54) BIORESPONSIVE POLYMER FORMULATIONS FOR DELIVERY OF BIOACTIVE AGENTS

(75) Inventor: Jennifer A. Neff, Rancho Santa Margarita, CA (US)

(73) Assignee: ALLVIVO VASCULAR, INC., Lake Forest, CA (US)

(21) Appl. No.: 12/980,293

(22) Filed: Dec. 28, 2010

Related U.S. Application Data
(63) Continuation-in-part of application No. 10/969,541, filed on Oct. 20, 2004, now Pat. No. 7,858,108.
(60) Provisional application No. 60/513,057, filed on Oct. 21, 2003.

(51) Int. Cl. A61K 9/14; A61P 17/02; A61P 41/00; C07K 17/08; A61K 47/48

(52) U.S. Cl. 424/484; 530/402; 424/78.17

(57) ABSTRACT

Elutable coatings and materials comprising protein resistant polymer components and bioactive agent on medical devices are disclosed. The elutable coatings comprise labile linkers that can be cleaved in response to a targeted physiologic stimulus. Medical devices formed from elutable materials comprised of protein resistant polymer components and bioactive agents are also disclosed. The elutable materials comprise labile linkers that can be cleaved in response to a targeted physiologic stimulus.

### CROSS-REFERENCE TO RELATED APPLICATION

This application is a continuation-in-part of U.S. application Ser. No. 10/969,541 filed Oct. 20, 2004, and entitled “Elutable surface coating”, which claims the benefit of U.S. Provisional Application No. 60/513,057, filed Oct. 21, 2003, which applications are incorporated by reference.

### BACKGROUND OF THE INVENTION

This invention relates to bioresponsive polymer formulations use as elutable coatings on medical devices as well as elutable gels and matricies for the purpose of delivering bioactive agents.

The systemic administration of drug agents, such as by intravenous means, treats the body as a whole even though the disease to be treated may be localized. Thus, it has become common to treat a variety of medical conditions by introducing an implantable medical device partly or completely into a body cavity within a human or veterinary patient. For example, many treatments of the vascular system entail the introduction of a device such as a stent, catheter, balloon, guide wire, cannula or the like. One of the potential drawbacks to conventional drug delivery techniques with the use of these devices being introduced into and manipulated through the vascular system is that blood vessel walls can be disturbed or injured. Clot formation or thrombosis often results at the injured site, causing stenosis (closure) of the blood vessel.

Other conditions and diseases are also treatable with stents, catheters, cannulae and other devices inserted into the esophagus, trachea, colon, biliary tract, urinary tract, sinus and nasal cavities, peritoneal cavity, eye and other locations in or on the body, or with orthopedic devices, implants, or replacements, for example.

Other drawbacks of conventional means of drug delivery using such devices is the difficulty in effectively delivering the bioactive agent over a short term (that is, the initial hours and days after insertion of the device) as well as over a long term (the weeks and months after insertion of the device). Another difficulty with the conventional use of devices for drug delivery purposes is providing precise control over the delivery rate of the desired bioactive agents, drug agents or other bioactive material.

### BRIEF SUMMARY OF THE INVENTION

It is desirable to develop devices and methods for reliably delivering suitable amounts of therapeutic agents, drugs or bioactive materials directly into a body portion during or following a medical procedure or injury, so as to treat or prevent such conditions and diseases, for example, to prevent abrupt closure and/or restenosis of a body portion such as a passage, lumen or blood vessel.

In view of the potential drawbacks to conventional drug delivery techniques, there exists a need for a device, method and method of manufacture which enable a controlled localized delivery of active agents, drug agents or bioactive material to target locations within a body.

One embodiment is a class of compounds for coating a medical device with the formula:

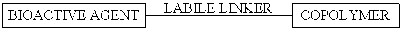

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

Another embodiment is a medical device comprising a class of compounds for coating the medical device with the formula:

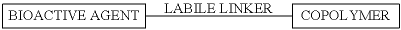

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

Another embodiment is a method of coating a medical device with a surface coating comprising a bioactive agent with the formula:

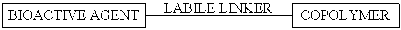

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

Another embodiment is a method of delivering a bioactive compound to a patient with a medical device with a surface coating comprising a bioactive agent with the formula:

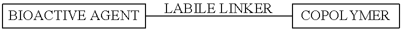

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

Another embodiment is a medical device that is in the form of a liquid, gel, foam or other matrix and is comprised of a bioactive agent with the formula:

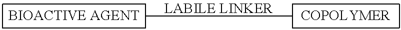

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect. In this embodiment, the bioactive agent with the above formula may be combined with a second polymer or polysaccharide to modify the physical properties and or stability of the gel, foam or matrix produced.

Another embodiment is a method of forming a medical device that is in the form of a liquid, gel, foam or other matrix and is comprised of a bioactive agent with the formula:

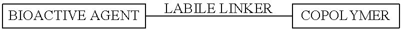

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect. In this embodiment, the bioactive agent with the above formula may be combined with a second polymer or polysaccharide to modify the physical properties and or stability of the gel, foam or matrix produced.

Another embodiment is a method of delivering a bioactive compound to a patient with a medical device that is in the form of a liquid, gel, foam or other matrix and is comprised of a bioactive agent with the formula:

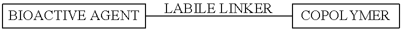

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect. In this embodiment, the bioactive agent with the above formula may be combined with a second polymer or polysaccharide to modify the physical properties and or stability of the gel, foam or matrix produced.

As used herein, the term “targeted physiologic stimulus” refers to changes in the tissue environment surrounding the implant, medical device, or bioactive agent delivery vehicle that causes the labile liner to be cleaved. Examples of such targeted physiologic stimulus include, but are not limited to, inflammation, coagulation, and infection. Both inflammation and infection will lead to an increase in activity of inflammatory cells. One way that this is manifested is an increase in certain types of proteases. Therefore, a protease susceptible labile linker may be selected to be susceptible to a specific type of protease that is released by inflammatory cells. In this manner, the device may release the bioactive agent only in response to an increase in the targeted protease. This effectively releases the bioactive agent in response to an increase in inflammation or infection, which is a biofeedback type response. Both inflammation and infection will also cause a drop in pH, which can cause an increase in hydrolysis. If a hydrolysable linker is used, then more of the bioactive agent will be released in response to an increase in inflammation or infection. Similarly, if specific proteins involved in the clotting cascade are targeted, then the labile linker can be selected to release an anticoagulant in response to the initiation of coagulation.

Other systems, methods, features, and advantages of preferred embodiments will be or become apparent to one with skill in the art upon examination of the following drawings and description. It is intended that all such additional systems, methods, features, and advantages be included within this description, be within the scope of preferred embodiments.

### BRIEF DESCRIPTION OF THE DRAWINGS

FIG. 1 is a graph showing activity of unmodified Factor H and Factor H derivatized with different concentrations of N-succinimidyl 3-(2-pyridyldithio)propionate (SPDP).

FIG. 2 is a graph showing relative absorbance as a result of Factor H being coupled to polystyrene (PS) in a dose dependent manner using end-group activated polymer (EGAP).

FIG. 3A is a graph showing relative absorbance as a result of Factor H being immobilized on polyether sulfone (PES).

FIG. 3B is a graph showing relative absorbance as a result of Factor H being immobilized on polyurethane (PU).

FIG. 3C is a graph showing relative absorbance as a result of Factor H being immobilized on polytetrafluoroethylene (PTFE).

FIG. 3D is a graph showing relative absorbance as a result of Factor H being immobilized on cellulose acetate (CA).

FIG. 3E is a graph showing relative absorbance as a result of Factor H being immobilized on polystyrene (PS).

FIG. 4 is a graph showing C3a levels in serum samples that were incubated with untreated PS, polystyrene coated with EGAP, PS coated with EGAP and incubated with native Factor H, or PS coated with EGAP and incubated with SPDP modified Factor H.

FIG. 5 is a graph showing results of EIA for Factor H bound to various substrates: (A) untreated stainless steel; (B) pretreated stainless steel; (C) stainless steel coated with Factor H; (D) pretreated stainless steel coated with Factor H; (E) pretreated stainless steel coated with F108 followed by Factor H; (F) pretreated stainless steel coated with EGAP followed by Factor H.

### DETAILED DESCRIPTION OF THE INVENTION

The disclosure relates to a coating comprising a bioactive compound for medical devices and methods for the controlled, localized delivery of a bioactive agent to target locations within a body. The disclosed embodiments also provide a material that may be in the form of a liquid, gel, foam, or other matrix, comprising a bioactive compound for the controlled, localized delivery of a bioactive agent to target locations within a body. The term “controlled localized delivery” as used herein is defined as a characteristic release rate of the bioactive agent at a fixed location.

In one non-limiting embodiment, a coating is applied to the device comprising a copolymer, a labile linker, and a bioactive agent. Hence, a coating of preferred embodiments provides a copolymer component for adhering to a medical device, a labile linker, and a bioactive agent, as shown below:

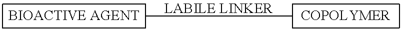

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

Another non-limiting embodiment includes a medical device comprising a class of compounds with the formula:

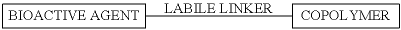

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect.

In another embodiment, a bioresponsive material is produced in the form of a liquid, gel, foam or other matrix comprising a copolymer, a labile linker, and a bioactive agent. The material serves as a type of medical device or a drug delivery vehicle. Hence, a material of a preferred embodiment is formed from a copolymer component, a labile linker, and a bioactive agent, as shown below:

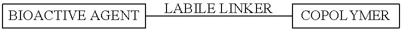

wherein the copolymer comprises one or more hydrophilic domains and at least one hydrophobic domain, labile linker is a linkage that can be cleaved in response to a targeted physiologic stimulus, and the bioactive agent is any agent such as a pharmaceutical agent or drug or other material that has a therapeutic effect. In this embodiment, the bioactive compound with the above formula may be combined with a second polymer to modify the physical properties and or stability of the material produced. In this embodiment, the material may behave as a liquid at room temperature or temperatures below body temperature and may behave as a solid at body temperature.

Some copolymers disclosed herein are temperature-responsive polymers, and depending on the concentration, are known to display a liquid form in aqueous solutions at lower temperatures that will form gels at higher temperatures. One non-limiting example of such copolymers are the class of copolymers known as PLURONICS, including (poly(ethylene oxide)-poly(propylene oxide)-poly(ethylene oxide) (PEO-PPO-PEO) and related copolymers. Because of this behavior, such copolymers have been studied as in situ gelling systems for injectable cell therapy and protein delivery. Solutions (typically greater than 16 wt %) of PLURONICS exhibit temperature-responsive sol-gel transition behaviors at a low critical solution temperature (LCST). This gelation behavior is primarily due to the formation of micelles through hydrophobic interactions. These spherical micelles are closely packed together above the LCST and strong physical interactions result in a drastic change in their rheological properties to form a gel. Different PLURONICS having different lengths of PEO and PPO chains will display different LCST. Therefore, one can combine different types of PLURONICS to fine tune the temperature at which a solution of a given concentration will gel. The higher the concentration of the copolymers, the more stable the gel they formed would be once injected in vivo. Example 17, describes the combination of EGAP with PLURONIC F68 to get a highly concentrated solution that would be liquid at room temperature and a solid at body temperature. One problem with these types of gels is that they tend to get dissolved or “wash out” once injected into a wound or body cavity too quickly. To enhance the stability of the gel, a second polymer may be added that can be crosslinked. This second polymer helps stabilize such gels for applications where they need to remain intact for longer time periods.

In certain embodiments, the surface to be coated is hydrophobic. Examples of preferred surfaces include, but are not limited to polystyrene (PS), polymethylmethacrylate (PMMA), polyolefins (e.g. polyethylene (PE), polypropylene (PP)), polyvinylchloride (PVC), silicones, polyacrylonitrile (PAN), copolymers of polyacrylonitrile/polyvinal chloride, polysulfones (e.g. polysulfone, poly(ether sulfone) (PES), polyphenylsulfone), acrylonitrile butadiene styrene (ABS), polyether block amide (PEBA), natural rubber, certain polyurethanes and polyurethane-poly(deimethylsiloxane), poly(tetrafluoroethylyene) (PTFE) and expanded PTFE, polyamide, polycarbonate, poly(ethylene terephthalate), polyimide and polyetherimide, poly(etheretherketone) (PEEK), poly(oxymethylene), pyrolized materials, and copolymers containing these constituents. Lesser hydrophobic materials and biodegradable materials are also contemplated by the preferred embodiments. These materials include, but are not limited to, polyvinyl acetate (PVAC), cellulose acetate, biodegradable polymers such as (PGA), polylactide (PLA), poly(lactic-glycolic acid) (PLGA) and their use with hydroxyapatite, tricalcium phosphate and trimethylenecarbonate, poly(ε-caprolactone, poly(dioxanone) (PDO), trimethylene carbonate, (TMC) polyaminoacids, polyesteramides, polyanhydrides, polyorthoesters and copolymers of these materials.

In certain embodiments, the surface to be coated is a tissue. The coating may be applied to a tissue within a living mammal, to a transplant or to a tissue based material that may be processed. The tissue may be autogeneic, allogeneic, zenogeneic or engineered.

The coating composition can also be used to coat metals including, but not limited to, stainless steel, nitinol, tantalum and cobalt chromium alloys. It is recognized that such materials may require a pretreatment to achieve stable bonding of the coating composition to the substrate. Such pretreatments are well known to those skilled in the art and may involve such processes as silanization or plasma modification. A coating is applied to the material in the form of a multiblock copolymer that contains one or more hydrophilic domains and at least one hydrophobic domain. The hydrophobic domain can be adsorbed to a hydrophobic surface by hydrophobic bonding while the hydrophilic domains can remain mobile in the presence of a fluid phase.

Preferred copolymer units for forming the copolymer coating of preferred embodiments include, but are not limited to, poly(amidoamine) and poly(propylene oxide) (PPO), poly(carboxybetaine) and poly(lactic-co-glycolic acid) (PLGA), stearyl-poly-N-vinylpyrrolidone and dextran-polycaprolactone, poly(ethyl ethylene phosphate) and poly(3-hydroxybutyrate), poly(acrylic acid) and polystyrene, polyisoprene and polystyrene, poly(L-amino acid) and poly(ester), poly(2-hydroxyethyl methacrylate) and poly(styrene), poly(n-butyl methacrylate) and poly(2-dimethylaminoethyl methacrylate), poly(n-butyl methacrylate) and poly(2-hydroxyethyl methacrylate), poly(vinyl alcohol) and poly(butyl acrylate), poly(styrene) and poly(acrylic acid), poly(acrylic acid) and poly(1-vinylpyrrolidone), poly(methacrylic acid and poly(styrene), poly(vinyl pyrrolidone) and poly(benzyloxytrimethylene carbonate), poly(3-hexylthiophene) and poly(2-ethyl-2-oxazoline), poly(propylene oxide) and poly(ethylene oxide)(PEO), poly(butadiene) and PEO, PEO and poly(N-acetylethyleneimine), PEO and phenyl boronic acid, PEO and polyurethane, PEO and polymethylmethacrylate, and PEO and polydimethyl sulfoxide, poly(N,N-dimethylaminoethylmethacrylate) and PEO, poly(ε-caprolactone) and PEO, poly(lactic-co-glycolic acid and PEO, N-isopropylacrylamide and PEO, polyL-lactide and PEO, poly(lysine) and PEO, poly(2-vinylpyridine) and PEO, poly(2-(diethylamino)ethyl methacrylate) and PEO, poly(isoprene) and PEO, poly(nitrobenzyl methacrylate) and PEO, poly(t-butyl acrylate) and PEO, poly(t-butyl methacrylate) and PEO, poly(2-hydroxyethyl methacrylate) and PEO, poly(4-vinyl pyridine) and PEO, poly(isobutylene) and PEO, poly(dimethylsiloxane) and PEO.

In the preceding pairs of copolymer units, preferably, the hydrophilic domain comprises PEO. Copolymers using copolymer units of this type and their application to coating materials to prevent protein adsorption have been described previously [25-30, 31, Han, 1993 #46].

In a certain embodiment, the copolymer comprises pendant or dangling hydrophilic domains, such as poly(ethylene oxide) (PEO) chains. The other domain(s) of the copolymer comprises a hydrophobic domain, such as a poly(propylene oxide) (PPO) chain. The preferred embodiments also contemplate the use of lesser hydrophobic materials and biodegradable materials for the other domain. Additionally, a linking group (R) is attached to the copolymer on one end adjacent to the hydrophilic domain to form an end-group activated polymer. For example, the end-group activated polymer may be in the form of any arrangement of the PEO and PPO blocks with the general formula:

(R-PEO)ₐ(PPO)_{b} (1)

where a and b are integers, are the same or different and are at least 1, preferably a is between 1 and 6, and b is between 1 and 3, more preferably a is 1 to 2, and b is 1. The polymeric block copolymer has a PEO (—C₂H₄—O—) content between 10 wt % and 80 wt %, preferably 50 wt % and 80 wt %, more preferably between 70 wt % and 80 wt %.

The PEO chains or blocks are of the general formula:

—(—C₂H₄—O—)ᵤ (2)

where u is the same or different for different PEO blocks in the molecule. Typically, u is greater than 50, preferably between 50 and 150, more preferably between 80 and 130. The PPO blocks are of the general formula;

—(—C₃H₆—O—)ᵥ (3)

where v may be the same or different for different PPO blocks in the molecule. Typically, v is greater than 25, preferably between 25 and 75, more preferably between 30 and 60.

The copolymers may be branched structures and include other structures (e.g. bridging structures, or branching structures) and substituents that do not materially affect the ability of the copolymer to adsorb upon and cover a hydrophobic surface. Examples include the following copolymers described in the following paragraphs.

In another embodiment, the end-group activated polymer of preferred embodiments is a derivative of a polymeric tri-block copolymer with pendant R groups, as in Formula (4), below. For example, these tri-block copolymers have a hydrophobic center block of polypropylene oxide and hydrophilic end blocks of polyethylene oxide with terminal R groups, and can be represented by the formula:

R—(—C₂H₄—O—)ₓ—(C₃H₆—O—)_{y}—(C₂H₄—O—)_{z}—H (4)

where y is between 25 and 75, preferably between 30 and 60, and x and z are preferably the same, but may be different, and are between 50 and 150, preferably 80 and 130. Certain types of these polymeric surfactants are commercially referred to as “PLURONIC™” or “POLOXAMERS™”, and are available, for example, from BASF.

Another suitable class of polymeric block copolymers is the di-block copolymers where a=1 and b=1, and can be represented by the formula;

R—PEO-PPO—H (5)

where PEO and PPO are defined above.

Another suitable class of polymeric block copolymers is represented by the commercially available TETRONIC™ surfactants (from BASF), which are represented by the formula:

(R—(O—C₂H₄)ᵤ—(O—C₃H₆)ᵥ)₂N—CH₂—CH₂—N((—C₃H₆—O—)ᵥ—(—C₂H₄—O—)ᵤ—H)₂ (6)

As used herein, the terms “PLURONIC” or “PLURONICS” refer to the block copolymers defined in Equation (1), which include the PLURONICS™ tri-block copolymer surfactants, the di-block surfactants, the TETRONIC™ surfactants, as well as other block copolymer surfactants as defined.

In the coatings of preferred embodiments, a labile linker connects the copolymer and the bioactive agent. The labile linker is a linkage which can be selectively cleaved upon exposure to a targeted physiologic stimulus. Non-limiting examples of a targeted physiologic stimulus include, but are not limited to, hydrolysis, radiation, ultrasound, enzymatic, ionic, diffusion, barrier-mediated diffusion, competitive displacement, and liposomal disruption. The structure of the labile linker depends on the mechanism of cleavage. As disclosed previously, a specific functional group is attached to the free end of a hydrophilic domain to form an end-group activated polymer. The specific functional group (R) eventually becomes a labile linker between the copolymer and bioactive agent in the preferred embodiments.

Protease susceptible linkers are particularly useful because many pathological conditions result in the production or upregulation of certain proteases. By linking a drug or therapeutic molecule to a medical device via a linker that is susceptible to cleavage by such a protease, it is possible to produce a device that releases a drug in response to the on set or changes in an adverse condition. In this way the device acts in a feedback mode and releases drug only when it is needed. It also provides a mechanism for releasing more or less drug in response to a change in the condition. Furthermore, it is possible to vary the amino acid residues that flank the cleavage sites of proteases in order to obtain sequences that will be degraded at lower or higher rates. This enables one to fine tune or control the feedback response of the drug eluting material for a particular condition by controlling the susceptibility of the linker to the environment in which the device will be placed.

As an example, the present invention would be useful for producing coated stents where the coating provides a mechanism for release of an anti-inflammatory in response to an increase in the inflammatory state of an artery. Inflammation is known to play a critical role in restenosis after stent implantation. Matrix metalloproteinases (MMP) are an important part of this process and play a key role in the arterial response to injury. MMP-9 is differentially upregulated as a result of arterial injury and has been found to substantially increase after stent implantation. The present invention would provide a means to link an antiinflammitory drug to the surface of the stent via a linker that is a substrate for MMP-9. After implantation, the antiinflammitory drug would be released if/when the tissue environment signals an unacceptable increase in the level of inflammation.

TABLE 1
Examples of linkers that can be cleaved by different mechanisms.

| Linker | Mechanism of Cleavage |
|---|---|
| Peptides containing either Gly-Ile or | Protease attack |
| Gly-Leu sequence | Target of some MMPs |
| GPQG-IAGQ | (upregulated in atherosclerosis |
| VPMS-MRGG (MMP-1 Consensus | and inflammation. |
| IPVS-LRSG (MMP-2 Consensus) | |
| RPFS-MIMG (MMP-3 Consensus) | |
| VPLS-LTMG (MMP-7 Consensus) | |
| VPLS-LYSG (MMP-9 Consensus) | |
| IPES-LRAG (MT1-MMP Consensus) | |
| PAPR-G | Thrombin |
| GR-G | |
| LDPR-S | |
| LVPR-GS | |
| AQCR-KYCP | Coagulation Factor Xa |
| | Coagulation Factor VIIa |
| TKPK-MLPP | Chymase |
| KPV-SDF | Cathepsin G |
| | Enzyme attack (bond cleavable |
| | by complement convertase) |
| Ester bond | Hydrolysis |
| NTA - Ni⁺⁺ - HHHHHH | Ionic |
| Metal chelator - metal ion - his tag | |
| Protein - protein or protein - peptide | |
| interactions that require a divalent ion | |
| | Competitive displacement |
| | Radiation |

In a preferred embodiment, the specific functional group (R) may contain a member of the reactive group, such as, hydrazine group, maleimide group, thiopyridyl group, tyrosyl residue, vinylsulfone group, iodoacetimide group, disulfide group or any other reactive group that is stable in an aqueous environment and that does not significantly impair the adsorption of the copolymer on the surface.

R can also comprise functional groups capable of forming ionic interactions with proteins, for example a nitrilotriacetic acid (NTA) group, which, when bound to a metal ion forms a strong bond with histidine tagged proteins. NTA modified PLURONICS are described in U.S. Pat. No. 6,987,452 to Steward et al., hereby incorporated by reference.

R may also comprise oligonucleotides that can bind to oligonucleotide tagged proteins. Oligonucleotide modified PLURONICS are described in PCT application No PCT/US02/03341 to Neff et al., hereby incorporated by reference.

In a preferred embodiment, the R group comprises a R′—S—S group where R′ is to be displaced for the immobilization of a bioactive agent. Therefore, the labile linker of the preferred embodiments comprises a disulfide bond. The substituent R′ is selected from the group consisting of (1) 2-benzothiazolyl, (2) 5-nitro-2-pyridyl, (3) 2-pyridyl, (4) 4-pyridyl, (5) 5-carboxy-2-pyridyl, and (6) the N-oxides of any of (2) to (5). A preferred end group is 2-pyridyl disulfide (PDS). The reactivity of these groups with proteins and polypeptides is discussed in U.S. Pat. No. 4,149,003 to Carlsson et al. and U.S. Pat. No. 4,711,951 to Axen et al, all of which are hereby incorporated by reference. As mentioned above, end group activated polymers (EGAP)s are generally a class of composition comprising a block copolymer backbone and an activation or reactive group.

Preferred embodiments can include the use of EGAP coatings for affecting delivering bioactive agents. In that respect, the second component of the coating of preferred embodiments can be a bioactive agent that is attached to the material through the activated end groups of the EGAP. The term “bioactive agent” is used herein to mean any agent such as a pharmaceutical agent or drug or other material that has a therapeutic effect. In general terms, a bioactive agent can be a pharmaceutical agent, protein, peptide, proteoglycan, oligonucleotide, protein fragment, protein analog, antibody, carbohydrate or other natural or synthetic molecule. Proteins can be acquired from either natural sources or produced recombinantly. Furthermore, the active domains of these proteins have been identified and recombinantly produced fragments that include these domains may be used. In a certain embodiment, more than one bioactive agent can be immobilized onto one surface with the use of EGAP material. The use of EGAP for protein immobilization has been described previously by Caldwell and others. However, Caldwell and others used EGAP to prepare biologically active surfaces for the purpose of evaluating or promoting specific protein-protein interactions and cell adhesion to surfaces [Neff, 1998 #12; Neff, 1999 #11; Webb, 2000 #8; Li, 1996 #15; Basinska, 1999 #21].

Alternatively, the bioactive agent component of the coating of preferred embodiments can be a therapeutic entity that is capable of removing specific components from a fluid. For example, to remove specific components from blood, the second component can be an antibody.

Bioactive agents that may be delivered using this invention include, but are not limited to, regulators of complement activation, including, Factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, oligonucleotide inhibitors of complement proteins, VCP and SPICE; regulators of coagulation or thrombosis, including, antithrombogenic agents, fibinolytic agents, thrombolytic agents, thrombin inhibitors, and antiplatelet agents; vasospasm inhibitors, vasodilators, antihypertensive agents, calcium channel blockers, antimitotics, microtubule inhibitors, actin inhibitors, antiproliferative agents, migration inhibitors, anticancer chemotherapeutic agents, antiinflammitory and immunosuppressive agents, growth factor and cytokine antagonists, growth factors, cytokines, chemotactic agents, gene therapy constructs, antisense oligonucleotides, antioxidants anti microbial agents, bactericidal agents, and anti viral agents.

The modified polymeric surfactant adsorbs with the hydrophobic domain of the copolymer upon the hydrophobic surface and the pendant hydrophilic domain of the copolymer and attached bioactive agent dangling away from the surface into the aqueous surroundings. Using a diblock copolymer as an example, the adsorbed surface can be illustrated by the formula below:

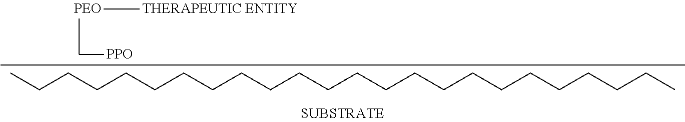

Preferred embodiments can be formed by dipcoating a substrate in a aqueous solution containing EGAP. The EGAP material is applied to the material in a solution of water, buffer, or a combination of water and an organic solvent, such as alcohol. Due to their amphiphilic nature, these copolymers will self assemble on hydrophobic materials from aqueous solutions. The hydrophobic block forms a hydrophobic bond with the material while the hydrophilic blocks remain mobile in the fluid phase. In this way, the hydrophilic chains form a brush like layer at the surface that prevents adsorption of proteins and cells. In certain embodiments, the coated substrate may be further treated by the application of irradiation, which may include UV, gamma or e-beam irradiation. In certain embodiments, the coated substrate may be further treated by the application of heat.

In certain embodiments, a medical device or drug delivery vehicle is formed from a copolymer component, a labile linker, and a bioactive agent, as shown below:

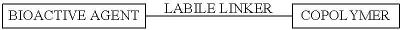

In this embodiment, the compound with the above formula may be combined with a second polymer, with or without the addition of cations, to modify the physical properties and or stability of the material (liquid, gel, foam or matrix) produced. The second polymer may enable the formation of crosslinked network within the material, which may be temporary or permanent, physical or chemical in nature. The crosslinks may be formed only between the second polymer molecules or they may be formed between the second polymer and the bioactive compound.

In one non-limiting embodiment, the second polymer is a polysacharide. A preferred polysaccharide is alginate which may be combined with cations, such as Calcium or Magnesium. Sodium alginate is a naturally occurring anionic polysaccharide that is made up of two types of monomers, mannuronic acid and guluronic acid. A typical alginate will contain blocks of homo polyguluronic, blocks of homo polymannuronic and block of alternating mannuronic and guluronic acids. Carboxyl groups from the uronic acid give it a negative charge. When calcium is added to sodium alginate, the calcium preferentially interacts with stretches of homoguluronic acid to form complexes that induce a parallel arrangement of complexed chains and instantaneous gel formation. The alginate is effectively crosslinked through strong ionic interactions in this way. The degree of crosslinking can be varied based on the relative concentration of alginate and cation ions to achieve a variety of properties. In one non-limiting embodiment, the second polymer is an EGAP where the terminal functional groups of EGAP are capable crosslinking. A preferred EGAP that is capable of crosslinking is EGAP having terminal diacrylate groups (EGAP-DA). In this embodiment, irradiation, which may be in the form of UV, gamma or e-beam may be applied to facilitate crosslinking. Alternatively, heat may be applied to facilitate crosslinking.

In certain embodiments, the copolymer-labile linker-bioactive agent construct is formulated to produce a medical device. The medical device may be administered to treat wounds. As used herein, the term “wound” and “wounds” include, but are not limited to tissue damage caused by surgery and accidental tissue injury, wounds that arise from various disorders including, but not limited to, autoimmune, allergic, infection, burns, diabetic, pressure, etc. Medical devices that may be produced include but are not limited to wound dressings, ulcer treatments, hemostatic dressings, products to treat aneurysms, products to prevent post surgical adhesions, products to improve post surgical healing including reducing scar formation, tissue repair or regeneration matrices including hernia repair or stoma reinforcement products, products for cosmetic surgery, and products for treatment of nerve, bone, and muscle defects. Such devices may be formed by dissolving the copolymer-labile linker-bioactive agent construct in an aqueous solution at a concentration sufficient to achieve gel formation at or near body temperature. The concentration selected will depend on the type of copolymer incorporated in the copolymer-labile linker-bioactive agent construct and the type of bioactive agent employed. It will also depend on the incorporation of a second polymer. The concentration may be selected to produce a liquid at room temperature which will transform to a gel at or near body temperature. Preferably, the concentration of the copolymer-labile linker-bioactive agent is in the range of 10% to 30%. More preferably, the concentration of the copolymer-labile linker-bioactive agent is in the range of 16% to 25%. These products may be formed into a desired shape and subsequently lyophilized. The lyophilized products may be used in the form of a pad, pellets, or powder. Lyophilized products may be applied in a dry form or may be reconstituted to a liquid or gel prior to application. These products may be formulated as a solution, foam or gel and may be delivered using an accessory device such as a syringe, spray device, or a catheter. Application of these products to a reinforcing or backing material such as a mesh or scaffold prior to application is also contemplated by the invention.

The function and treatment potential of the devices listed are substantially enhanced by the delivery of the bioactive agent. Therefore, delivery or application of the above devices provides a means for delivery of the bioactive agent to the mammal. Bioactive agents that may be delivered using this invention are described herein.

In certain embodiments, the copolymer-labile linker-bioactive agent construct is used to produce a drug delivery vehicle. Drug delivery vehicles that may be produced include but are not limited to: ocular drug delivery vehicles, microparticles or gels that are placed within dental pockets for treatment of periodontal disease, and intraperitoneal therapy.

Advantages of preferred embodiments include the use of a non-hazardous coating method, no harsh environmental conditions, no toxic chemicals and no toxic waste products. Preferred embodiments incorporate a simple coating method that is readily incorporated in production process and does not require highly skilled personnel.

The compositions of preferred embodiments can be used for any medical device. The term “medical device” appearing herein is a device having surfaces that contact human or animal bodily tissue and/or fluids in the course of their operation. The definition includes, but is not limited to, endoprostheses implanted in blood or tissue contact in a human or animal body such as balloon catheters, A/V shunts, vascular grafts, stents, pacemaker leads, pacemakers, heart valves, and the like that are implanted in blood vessels or in the heart. The definition also includes within its scope devices for temporary intravascular use such as catheters, guide wires, and the like which are placed into the blood arteries or vessels or the heart for purposes of monitoring or repair. The definition also includes neuroprothetics, orthopedic, dental, and optical devices. The medical device can be intended for permanent or temporary implantation. Such devices may be delivered by or incorporated into intravascular and other medical catheters or delivery devices.

The compositions of preferred embodiments can be used for any device used for ECC. As stated above, ECC is used in many medical procedures including, but not limited to, cardiopulmonary bypass, plasmapheresis, plateletpheresis, leukopheresis, LDL removal, hemodialysis, ultrafiltration, and hemoperfusion. Extracorporeal devices for use in surgery include blood oxygenators, blood pumps, blood sensors, tubing used to carry blood and the like which contact blood which is then returned to the patient.

The compositions of preferred embodiments may be used to produce medical devices that are used to treat wounds, repair, replace or regenerate tissue. They may also be used to produce medical devices that are applied solely to deliver therapeutics.

The disclosure below is of specific examples setting forth preferred methods. The examples are not intended to limit scope, but rather to exemplify preferred embodiments.

### Example 1(A)

### Preparation of Substrate with Releasable RCA—Method 1

PLURONIC F108 is derivatized to incorporate a terminal PDS group as described by Li et al. This pyridyl disulfide activated PLURONIC (EGAP-PDS) is dissolved in phosphate buffer, pH 7.4, 1 mM EDTA (PB) and then mixed with a polypeptide having the sequence CGPQG-IAGQ. The reaction is allowed to proceed for 2 hours at room temperature and is monitored by measuring the release of pyridyl 2-thione spectrophotometrically at 343 nm. The product is purified by dialysis and recovered by lyophilization. Multiple reactions are performed using this approach where the ratio of peptide to EGAP-PDS is varied and the ratio that produces that highest degree of EGAP derivitization is determined.

EGAP modified with the protease susceptible linker (EGAP-PSL) as described above is dissolved in PB. The substrate or device to be coated is incubated with this solution for 30 minutes to overnight. The coated substrate is washed and then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. After completion of the reaction, hydroxylamine (10 mM) is added to hydrolyze and deactivate any NHS remaining on the surface.

The amount of RCA on the surface is determined by enzyme immunoassay (EIA).

### Example 1(B)

### Preparation of Substrate with Releasable RCA—Method 2

A 1% solution of EGAP-PDS in PB is prepared. The substrate to be coated is immersed in this solution for 30 minutes to overnight. The coated substrate is washed with PB and then incubated with a polypeptide having the sequence CGPQG-IAGQ. The reaction is allowed to proceed for 2 hours at room temperature. The substrate is washed with PB.

The modified substrate is washed and then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. After completion of the reaction, hydroxylamine (10 mM) is added to hydrolyze and deactivate any NHS remaining on the surface.

The amount of RCA on the surface is determined by enzyme immunoassay (EIA).

### Example 1(C)

### Preparation of Substrate with Releasable RCA—Method 3

A copolymer of PEO-polybutadiene-PEO is derivatized to incorporate a terminal PDS group as described by Li et al. This pyridyl disulfide activated PLURONIC (EGAP-PDS) is dissolved in phosphate buffer, pH 7.4, 1 mM EDTA (PB) and then mixed with a polypeptide having the sequence CGPQG-IAGQ. The reaction is allowed to proceed for 2 hours at room temperature and is monitored by measuring the release of pyridyl 2-thione spectrophotometrically at 343 nm. The product is purified by dialysis and recovered by lyophilization. Multiple reactions are performed using this approach where the ratio of peptide to EGAP-PDS is varied and the ratio that produces that highest degree of EGAP derivitization is determined.

A 1% solution of EGAP-PDS in PB is prepared. The substrate to be coated is immersed in this solution for 30 minutes to overnight. The coated substrate is washed with PB and then incubated with a polypeptide having the sequence CGPQG-IAGQ. The reaction is allowed to proceed for 2 hours at room temperature. The coated substrate is treated with UV irradiation and then washed with PB.

The modified substrate is washed and then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. After completion of the reaction, hydroxylamine (10 mM) is added to hydrolyze and deactivate any NHS remaining on the surface.

The amount of RCA on the surface is determined by enzyme immunoassay (EIA).

### Example 1(D)

### Preparation of Substrate with Releasable RCA—Method 4

A copolymer of PEO-polybutadiene-PEO is derivatized by reaction with p-nitrophenyl chloroformate to incorporate a terminal nitrophenyl group group as described by Li et al. This O-nitrophenyl activated PEO-polybutadiene-PEO (EGAP-ONP) is dissolved in dry methanol and then mixed with a polypeptide having the sequence CGPQG-IAGQ. The reaction is allowed to proceed overnight at room temperature and is monitored by measuring the absorbance at 402 nm. The product is purified by dialysis and recovered by lyophilization.

A 1% solution of EGAP-CGPQG-IAGQ in PB is prepared. The substrate to be coated is immersed in this solution for 30 minutes to overnight. The coated substrate is treated with UV irradiation and then washed with PB.

The modified substrate is then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. After completion of the reaction, hydroxylamine (10 mM) is added to hydrolyze and deactivate any NHS remaining on the surface.

The amount of RCA on the surface is determined by enzyme immunoassay (EIA).

### Example 2(A)

### Preparation of Substrate with Releasable RCA—Method 1

PLURONIC F108 is derivatized to incorporate a terminal NTA group as described by Ho et al. This pyridyl disulfide activated PLURONIC (EGAP-NTA) is dissolved in 25 mM NiSO₄ and incubated for 30 minutes. The Ni⁺⁺ charged EGAP-NTA (EGAP-NTA-Ni) is recovered and excess NiSO₄ is removed using a size exclusion column. EGAP-NTA-Ni is then mixed with a polypeptide having the sequence HHHHHHGPQG-IAGQ. The reaction is allowed to proceed for 2 hours at room temperature. The product is purified by dialysis and incubated with the substrate or device to be coated for 30 minutes to overnight. The coated substrate is washed and then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE.

The amount of RCA on the surface is determined by enzyme immunoassay (EIA).

### Example 2(B)

### Preparation of Substrate with Releasable RCA—Method 2

A 1% solution of EGAP-NTA is prepared in purified water. The substrate to be coated is immersed in this solution for 30 minutes to overnight. After washing the substrate is incubated with 25 mM NiSO₄ for 30 minutes at room temperature. The substrate is washed and then incubated with a polypeptide having the sequence HHHHHHGPQG-IAGQ for 2 hours at room temperature. The modified substrate is washed and then incubated with a mixture of EDC and N-hydroxysuccinimide in 4-morpholinoethanesulfonic acid (MES) for 15 minutes at room temperature to convert the carboxy terminus of the peptide to an amine reactive group. The substrate is washed and then incubated with the RCA protein or peptide of interest dissolved in MES for two hours at room temperature. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE.

The amount of RCA on the surface is determined by enzyme immunoassay (EIA).

### Example 3

### Characterization of Protease Induced Cleavage of the Suceptible Linker

Polystyrene microsphere samples are coated with the EGAP-PSL construct as described in either Example 1A or Example 1B. The coated microsphere samples are incubated with solutions containing a Matrix Metalloproteinase (MMP-1) immersed.

### Example 4

### Characterization of Protease Induced Cleavage of the Suceptible Linker and Release of RCA

A device or substrate is coated with the EGAP-PSL-RCA construct and described in either Example 1A or Example 1B. The coated substrate is incubated with a solution containing a Matrix Metalloproteinase (MMP-1) immersed.

### Example 5

### Immobilization of Recombinant RCA-PSL Construct on Substrate Coated with EGAP-PDS

A protein or peptide RCA is recombinantly expressed or synthesized, respectively, to contain a protease susceptible linker (PSL) at its N-terminus. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. The protease susceptible linker will contain a sequence that is a protease target for cleavage and at least one cysteine residue near its N-terminus. An example of such a peptide sequence is CGPQG-IAGQ, which is a target of matrix metalloproteinases. A substrate is coated with EGAP-PDS and then incubated with the RCA-PSL construct in PB for 2 hours at room temperature. The substrate is washed and the amount of RCA bound to the surface is measured by EIA.

### Example 6

### Immobilization of Recombinant RCA-PSL Construct on Substrate Coated with EGAP-NTA

A protein or peptide RCA is recombinantly expressed or synthesized, respectively, to contain a protease susceptible linker (PSL) at its N-terminus. The RCA may be any one of the following: factor H, factor H like protein 1 (FHL-1), factor H related proteins (FHR-3, FHR-4), C4 binding protein (C4bp), complement receptor 1 (CR1), decay-accelerating factor (DAF), membrane cofactor protein (MCP), compstatin, monoclonal antibody inhibitors of complement proteins, VCP or SPICE. The protease susceptible linker will contain a sequence that is a protease target for cleavage and a polyhistidine tag at its N-terminus. An example of such a peptide sequence is HHHHHHGPQG-IAGQ, which is a MMP target with a six His tag. A substrate is coated with EGAP-NTA and then incubated with 50 mM NiSO4 for 30 minutes at room temperature. The substrate is washed and then incubated with the RCA-PSL construct in PB for 2 hours at room temperature. The substrate is washed and the amount of RCA bound to the surface is measured by EIA.

### Example 7

### Immobilization of Factor H on Substrate with EGAP

Factor H is coupled to a substrate or device that is coated with EGAP-PDS. Factor H contains numerous cysteine residues, some of which may serve as sites for coupling via the PDS groups [33]. The combination of Factor H and EGAP on the surface of the substrate or device acts to down regulate complement activation.

A device or substrate is coated with Factor H by covering the device surface with a solution containing 0.1 to 4% of EGAP in water or water containing buffer salts. This may be accomplished using a dip coating method, for example. After a coating period of 30 minutes to 24 hours, the substrate is washed using water or buffer. Factor H is diluted into phosphate buffer, pH 7.5, and then added to the coated substrate. After and incubation period of 2-24 hours, the substrate is washed with buffer. The following controls are prepared for comparison: (1) The substrate is coated with unmodified F108 and subsequently incubated with Factor H and washed as indicated above, (2) The substrate is not treated with any initial coating but is incubated with Factor H and washed as indicated above, (3) The substrate is coated with unmodified F108 only, and (4) The substrate is left untreated. The amount of Factor H that is bound to each surface is determined by enzyme immunoassay using a commercially available biotinylated anti-factor H in conjunction with HRP modified streptavidin for detection.

Each substrate is evaluated to determine the ability of the surface bound factor H to inhibit complement activation when it comes into contact with whole blood, plasma or serum. To accomplish this, two types of assays are performed; one being an analysis of the surface to determine what has stuck to it and the other being an analysis of the blood to determine if specific proteins involved in the complement cascade have been activated. The amount of C-3 fragments that are bound to the substrate are determined by enzyme immunoassay (EIA). The amounts of fluid phase C3a, C1s-C1NA, and sC5b-9 complexes that are generated as a result of surface contact between the blood and the substrate are monitored using EIA.

In a previous study, it was found that Factor H could be applied to materials to down regulate complement activation. However, the method used to conjugate factor H to the material was, in of its self, complement activating. Coating a material with EGAP material produces the necessary sites for conjugating Factor H, however, it does not promote compliment activation. To the contrary, it produces a surface that is less biologically active than Polystyrene (PS) and most other materials to which it would be applied for blood contacting devices.

It is anticipated that it will be possible to bind higher amounts of biologically active Factor H to material surfaces than has previously been achieved using alternative methods. A previous study compared the amounts of Factor H bound to surfaces that displayed either pyridyl disulfide groups or sulfhydryl groups. Both surfaces were prepared by reacting a polyamine modified PS with N-succinimidyl 3-(2-pyridyldithio) propionate (SPDP) and the latter was obtained by subsequently treating the surface with dithiothreitol (DTT). It was found that greater amounts of Factor H bound to the material that was modified with SPDP only. In spite of this, the overall biological activity was lower. These results suggest that the conformation of Factor H on the two surfaces differed and that the SPDP modified surface caused a decrease in the biological activity of bound Factor H. PDS groups are more reactive toward free cysteines in factor H and could result in greater coupling efficiency. However, the SPDP modified surface, is also likely to be more hydrophobic and for this reason, it could result in greater amounts of nonspecifically bound proteins as well as a decrease in Factor H activity due to strong interfacial forces between the protein and the material. Using the EGAP approach described herein, it is possible to incorporate PDS groups at the material surface and thereby, achieve high coupling efficiencies without producing a hydrophobic or potentially denaturing surface.

Tethering Factor H to materials using EGAP decreases steric hindrance by incorporating a flexible spacer between the protein and the material. This makes it more accessible for binding to target proteins in blood or plasma.

The EGAP coating produces a highly hydrated brushlike layer at the material surface that effectively buffers the Factor H from the material. This prevents denaturation and preserves the native protein conformation and activity.

The EGAP coating prevents nonspecific protein adsorption. In blood and plasma there are many proteins that when adsorbed onto an artificial material can promote complement activation. For example, when fibrinogen adsorbs onto a material surface, it changes conformation such that it signals for the activation of EGAP prevents this type of interaction and thereby minimizes the risk of immune system activation. When combined with Factor H, the system prevents initial activation and then incorporates a backup, being Factor H that can down regulate any activation that might occur during an ECC procedure.

### Example 8

### Derivatization of Factor H to Incorporate Sulfhydryl Reactive Group

Factor H was incubated with various concentrations of N-succinimidyl 3-(2-pyridyldithio) propionate (SPDP) ranging from 7 to 67% at room temperature for 1 hour. Unbound SPDP was removed by dialysis. The activities SPDP modified factor H samples were measured and compared to that of unmodified factor H by measuring the ability of factor H to act as a cofactor to factor I. Factor I is another regulator of complement activation that inactivates C3b by cleaving it into inactive C3b (iC3b) and then into C3c and C3dg. This function of factor I is dependent on the presence of active factor H. The activities of the various solutions of modified factor H were thus determined by combining them with C3b and factor I and subsequently measuring the levels of degradation of C3b as follows: Aliquots of 10 μg C3b and 0.6 μg factor I were incubated together with factor H samples in the concentrations of 0.5, 1 and 2, μg for 60 min at 37° C. The reactions were terminated by boiling the samples in reducing electrophoresis sample buffer. The samples were then run on SDS-PAGE. An aliquot containing 10 μg of undigested C3b was added as a control to each gel. The gels were Coomassie stained, scanned and the amount of undigested alfa-prime chain of C3b in each sample was evaluated using NIH-image quant.

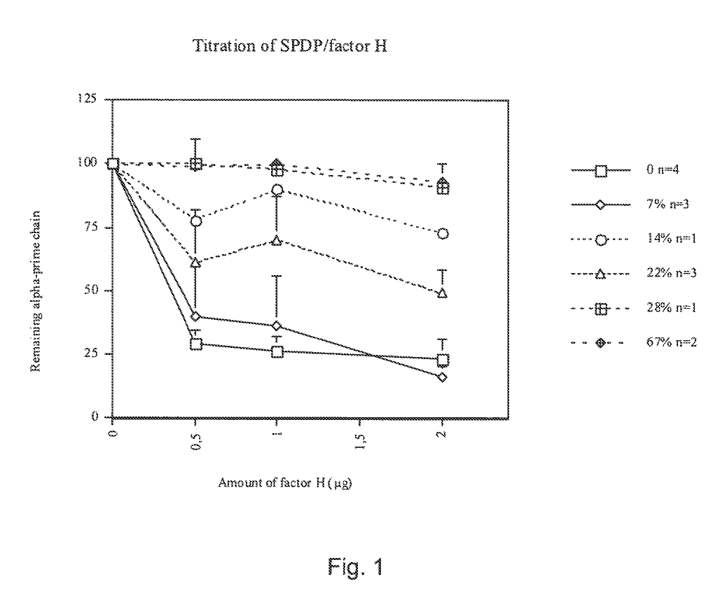

The results are shown in FIG. 1. The ratio of SPDP to factor H and the number of samples tested for each data point are given in the legend. The results indicate that Factor H is unaffected after treatment with 7% SPDP, but loses its activity gradually at higher concentrations. At 28% SPDP or higher, a totally inactive factor H is obtained, while concentrations between 25% and 7% yield partial inactivation.

### Example 9A

### Immobilization of Factor H on Substrate with EGAP and Heterobifunctional Crosslinker

Factor H is activated using a heterobifunctional crosslinker and then coupled to a substrate or device that is coated with EGAP. The combination of Factor H and EGAP on the surface of the substrate or device acts to down regulate complement activation.

A device or substrate is coated with Factor H by covering the device surface with a solution containing 0.1 to 4% of EGAP in water or buffer. This may be accomplished using a dip coating method, for example. After a coating period of 30 minutes to 24 hours, the substrate is washed using water or water containing buffer salts. Factor H is activated using a heterobifunctional crosslinker that is reactive towards amine groups, for example, and that incorporates a functional group that can be used to couple directly to the pyridyl disulfide group (PDS) present on EGAP. One such commercially available crosslinker is N-succinimidyl 3-(2-pyridyldithio) propionate (SPDP). The crosslinker incorporates pyridyl disulfide groups on the protein that can be reduced to yield sulfhydryl groups that will react directly with EGAP. Factor H is reacted with SDPD in phosphate buffer, pH 7.5 for 30-60 minutes and then purified using a PD-10 column. The activated protein is treated with 25 mM DTT in acetate buffer, pH 4.5. It is purified using a PD-10 column where it is also exchanged into phosphate buffer, pH 7.5. The product is incubated with the EGAP coated substrate for a period of 2-24 hours followed by washing with buffer. Controls are prepared as described in Example 1. The amount of Factor H that is bound to the surface is determined by enzyme immunoassay using a commercially available biotinylated anti-factor H in conjunction with HRP modified streptavidin for detection.

The modified substrate is evaluated to determine the ability of the surface bound factor H to inhibit complement activation when it comes into contact with whole blood, plasma or serums described in Example 1.

### Example 9B

### Immobilization of Factor H on Substrate with EGAP and Heterobifunctional Crosslinker

Factor H was activated using a heterobifunctional crosslinker, SPDP, and then coupled to an EGAP coated substrate. Using EGAP, it was possible to immobilize factor H in a dose dependant manner.

Substrates were coated with Factor H by covering them with a solution containing 1% of EGAP in water. After a coating period of 24 hours, substrates were washed with water. Control samples were prepared by substituting PLURONIC F108 for EGAP using the same procedure. Factor H was activated using a heterobifunctional crosslinker that is reactive towards amine groups and that incorporates a functional group that can be used to couple directly to the pyridyl disulfide group (PDS) present on EGAP. In this example, N-succinimidyl 3-(2-pyridyldithio) propionate (SPDP) was used. Factor H was reacted with SDPD in PBS, pH 7.5 for 30-60 minutes and then purified using a PD-10 column. The crosslinker effectively incorporated pyridyl disulfide groups on the protein. The EGAP coated surface was reduced by incubation with 25 mM DTT for 30 minutes and then washed taking care not to expose the surface to air. Immediately after washing, the substrate was reacted with different concentrations of the SPDP modified factor H for a period of 2-24 hours and finally, washed with buffer. The amount of Factor H that was bound to the surface was determined by enzyme immunoassay using a biotinylated anti-factor H in conjunction with HRP modified streptavidin for detection.

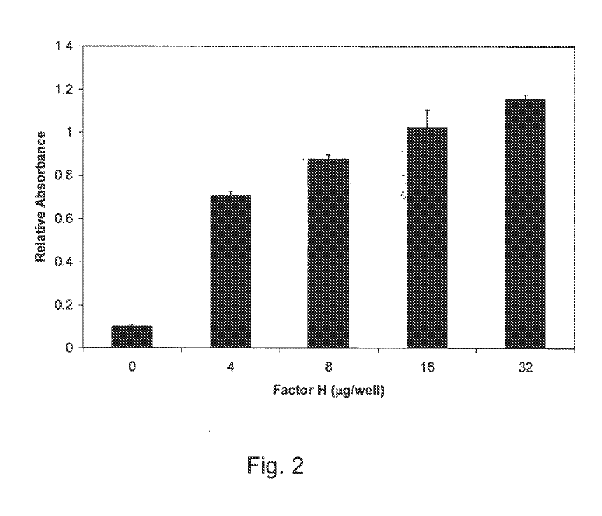

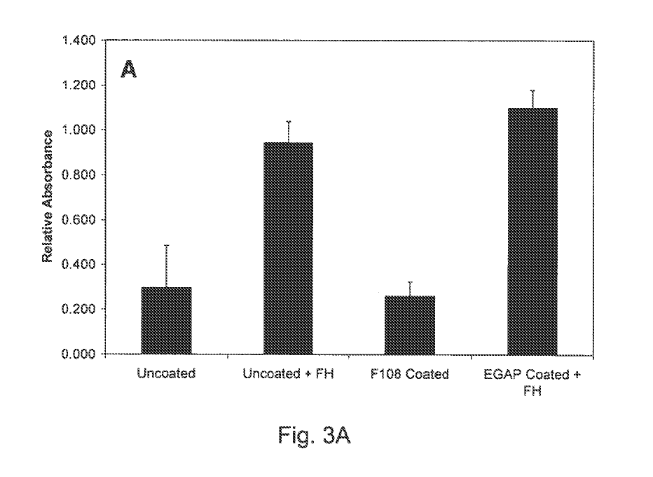

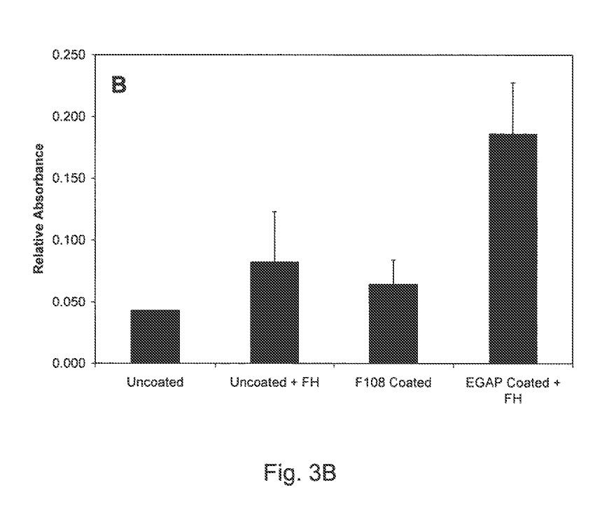

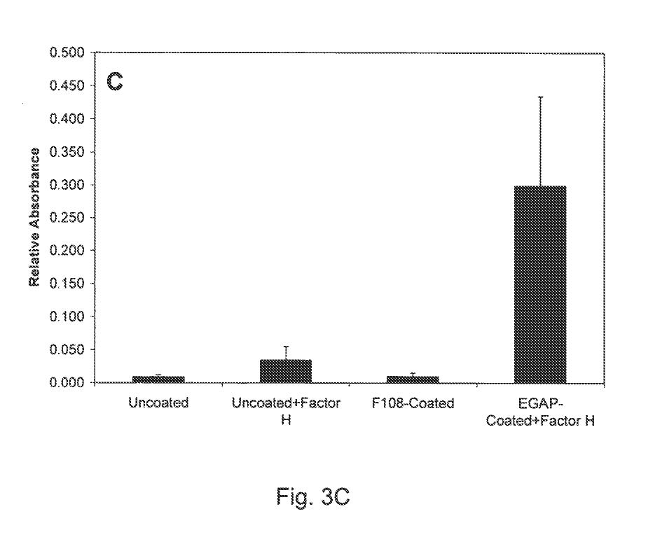

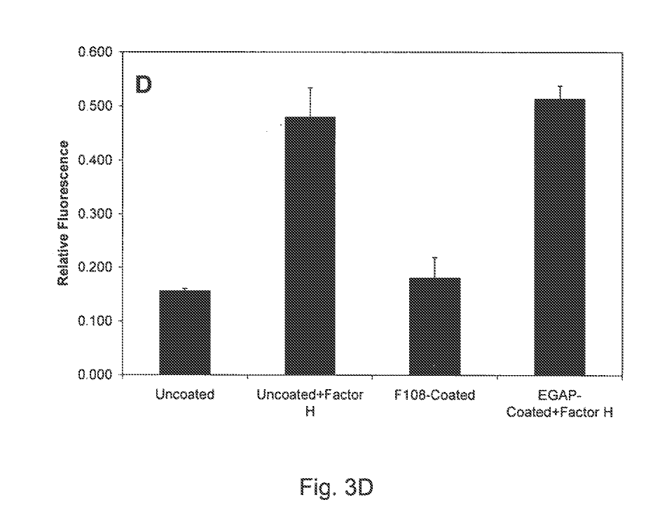

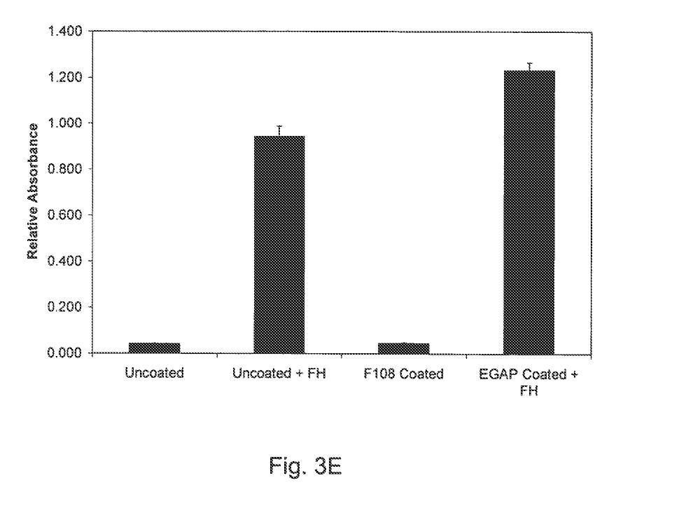

The results are shown in FIG. 2 and indicate that factor H is effectively bound to the surface in a dose dependant manner. Based on the low levels of factor H bound to F108 coated control samples (see FIG. 3 (E)), it is clear that the coupling to EGAP-coated surfaces is specifically mediated by functional groups on EGAP.

In a previous study, it was found that Factor H could be applied to materials to down regulate complement activation. However, the method used to conjugate factor H to the material was, in of its self, complement activating. Coating a material with EGAP produces the necessary sites for conjugating Factor H, however, it does not promote compliment activation. To the contrary, it produces a surface that is less biologically active than Polystyrene (PS) and most other materials to which it would be applied for blood contacting devices.

It is anticipated that it will be possible to bind higher amounts of biologically active Factor H to material surfaces using EGAP than has previously been achieved using alternative methods. A previous study compared the amounts of Factor H bound to surfaces that displayed either pyridyl disulfide groups or sulfhydryl groups. Both surfaces were prepared by reacting polyamine modified PS with N-succinimidyl 3-(2-pyridyldithio) propionate (SPDP) and the latter was obtained by subsequently treating the surface with dithiothreitol (DTT). It was found that greater amounts of Factor H bound to the material that was modified with SPDP only. In spite of this, the overall biological activity was lower. These results suggest that the conformation of Factor H on the two surfaces differed and that the SPDP modified surface caused a decrease in the biological activity of bound Factor H. PDS groups are more reactive toward free thiols in factor H and could result in greater coupling efficiency. However, the SPDP modified surface, is also likely to be more hydrophobic and for this reason, it could result in greater amounts of nonspecifically bound proteins as well as a decrease in Factor H activity due to strong interfacial forces between the protein and the material. Using the EGAP approach described herein, it is possible to incorporate functional groups at the material surface with very good reactivity and thereby, achieve high coupling efficiencies without producing a hydrophobic or potentially denaturing surface.

Tethering Factor H to materials using EGAP decreases steric hindrance by incorporating a flexible spacer between the protein and the material. This makes it more accessible for binding to target proteins in blood or plasma. Furthermore, the EGAP coating produces a highly hydrated brush like layer at the material surface that effectively buffers the Factor H from the material. This prevents denaturation and preserves the native protein conformation and activity.

### Example 10

### Immobilization of Factor H Using EGAP and SATA Crosslinker

Factor H was activated using a heterobifunctional crosslinker, SATA, and then coupled to a substrate or device that was coated with EGAP. The EGAP-factor H coating was effectively applied to various types of materials including polystyrene, polyether sulfone (PES), cellulose acetate (CA), polytetrafluoroethylene (PTFE), silicone, and polyurethane (PU).

Substrates or devices were coated with Factor H by covering the surface with a solution containing 1% EGAP in water. Control samples were prepared by substituting PLURONIC F108 for EGAP using the same procedure. Uncoated (UN) samples were also included for comparison. After a coating period of 24 hours, the substrates were washed with buffer. Factor H was activated using a heterobifunctional crosslinker, N-succinimidyl S-Acetylthioacetate (SATA) (Pierce Scientific). The N-hydroxysuccinimide (NHS) ester portion of this crosslinker reacts with amine groups on factor H and incorporates a protected sulfhydryl group that can be used to couple directly to the pyridyl disulfide group present on EGAP. SATA was dissolved in DMSO and then reacted with Factor H in PBS, pH 7.5 for 30-60 minutes. The activated factor H was purified using a PD-10 column. The modified groups on factor H were then deacetylated to remove the protecting group by treatment with hydroxylamine. A final purification on a PD-10 column was performed. EGAP coated substrates were incubated with the modified factor H overnight and then washed with buffer. The amount of Factor H that was bound to the surface was determined by enzyme immunoassay using a biotinylated anti-factor H in conjunction with HRP modified streptavidin for detection. The results are shown in FIG. 3 below and indicate that the EGAP-factor H coating was effectively applied to various types of materials including, polyether ether sulfone (PES), polyurethane (PU), polytetrafluoroethylene (PTFE), cellulose acetate (CA), and polystyrene (PS).

### Example 11

### Reduced Complement Activation on Substrate Coated with EGAP and Factor H

### Complement Activation is Measured by Production of C3a

Factor H was activated using a heterobifunctional crosslinker and then coupled to an EGAP coated substrate. Coated substrates and controls were incubated with human serum and the level of complement activation was accessed by measuring the amount of C3a generated. EGAP-Factor H coated substrates produced less complement activation compared to controls. Furthermore, both EGAP and F108 coated substrates produced less complement activation than untreated substrates.

A 96 well polystyrene plate was coated with Factor H by adding 300 □L of 1% EGAP in PBS to each well and placing the plate on a shaker at room temperature overnight. After coating, the substrate was washed with PBS. Factor H was reacted with 3.5% w/w SPDP in PBS, pH 7.5 for 1 hour and then purified by dialysis. The EGAP coated substrate was treated with 25 mM DTT for 1 hour. The DTT was removed and the plate was washed with PBS/EDTA pH 6.0 taking care not to expose the substrate to air. After washing, the substrate was immediately reacted with the SPDP activated factor H (100 □g/mL) overnight at 4° C. The factor H solution was removed and the substrate was washed with PBS. The following substrates were used as controls: untreated PS, polystyrene coated with F108 (results not shown), PS coated with EGAP, and PS coated with EGAP followed by incubation with native factor H. All substrates were incubated with human serum for different time periods up to one hour. At the end of each incubation period, EDTA was added to the serum to stop any further complement activation. The amount of C3a in each serum sample was measured by enzyme immunoassay.

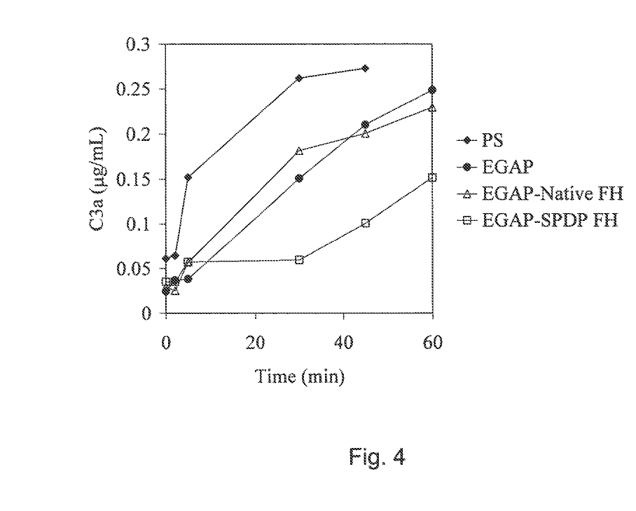

The results are shown in FIG. 4 below and indicate that the EGAP-Factor H coating effectively inhibits the generation of C3a compared to controls. Furthermore, the EGAP coating alone reduced the generation of C3a compared to the naked substrate.

### Example 12

### Immobilization of Factor H on Stainless Steel and Nitinol

Factor H was activated using a heterobifunctional crosslinker, SATA, and then coupled to a stainless steel device that was pretreated followed by coating with EGAP. Factor H was effectively bound to stainless steel via EGAP.

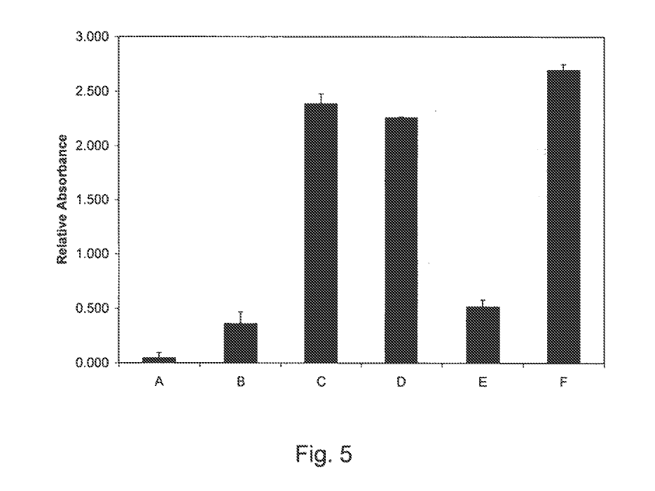

Stainless steel and nitinol stent devices were cleaned and/or pretreated followed by coating with EGAP and factor H as described in Example 4. Control samples were prepared by substituting PLURONIC F108 for EGAP using the same procedure. Factor H was activated using SATA as described in Example 4. EGAP coated substrates were incubated with the modified factor H overnight and then washed with buffer. The amount of Factor H that was bound to the surface was determined by enzyme immunoassay as described in Example 4. The results for stainless steel are shown in FIG. 5 and indicate that the EGAP-factor H coating was effectively applied to the metal substrate. Furthermore, based on the low amount of H measured on the F108 coated stainless, it is clear that the binding to EGAP coated substrates is specifically mediated by the PDS functional group on EGAP.

### Example 13

### Immobilization of Factor H on Substrate with EGAP and Unmodified F108

Factor H is coupled to a substrate or device that is coated with a combination of EGAP and unmodified F108. The ratio of EGAP to unmodified F108 is varied in order to vary the number of reactive sites for Factor H coupling and, in turn, vary the surface density of Factor H on the substrate or device. The optimal density of Factor H is determined by measuring the substrate's ability to down regulate complement activation. Although it is likely that the highest density of Factor H possible is optimal for this system, many potentially interesting peptides and synthetic regulators of complement may have some beneficial effects but also possibly some adverse or unknown effects on related blood components including platelets and leukocytes. This EGAP approach potentially provides an optimal system for determining such interactions and how concentrations effect such interactions. Furthermore, the protein, whether produced recombinantly or by purification from natural sources, is the most expensive component of the coating. For this reason, it is beneficial to determine the least amount of protein that can be used to achieve the desired level of performance. This system provides a means to effectively determine this level and subsequently reproduce this level with a high level of confidence.

A series of solutions containing the following ratios of F108 to EGAP are prepared in PBS where the total concentration of surfactant is 1%: (0:100, 5:95, 10:90, 25:75, 50:50, 75:25, 100:0). Substrates are coated with these solutions for a period of 24 hours, followed by washing with PBS. Factor H is diluted into phosphate buffer, pH 7.5, and then added to the coated substrate. After and incubation period of 2-24 hours, the substrate is washed with buffer. The amount of Factor H that is bound to each substrate is determined by enzyme immunoassay using a commercially available biotinylated anti-factor H in conjunction with HRP modified streptavidin for detection.

Each substrate is evaluated to determine the ability of the surface bound factor H to inhibit complement activation when it comes into contact with whole blood, plasma, or serum as described in Example 5.

### Example 14

### Immobilization of Two or More Therapeutic Entities on Substrate with EGAP

In this example, two or more therapeutic entities are immobilized on a substrate or device using EGAP where each entity affects a different component of the immune or haemostatic system. For example, a regulator of complement might be combined with a regulator of coagulation. EGAP provides a simple method for coimmobilizing two such factors and potentially enables one to control the ratio and densities of the factors, which may very well be critical in the delivery of two or more therapeutic agents from the solid phase.

Two or more types of EGAP are prepared where the end group activation process yields different types of terminal functional groups. These are referred to as EGAP-A and EGAP-B. Two or more therapeutic entities, referred to as TA and TB, are modified to react preferentially with EGAP-A and EGAP-B, respectively. EGAP-A and EGAP-B are combined in a predetermined ratio in PBS where the total concentration of EGAP is 1%. Substrates are coated with these solutions for a period of 24 hours, followed by washing with PBS. If the buffer conditions required for coupling TA to EGAP-A are the same as those required for coupling TB to EGAP-B, then TA and TB are diluted into buffer and added to the coated substrate simultaneously. If different buffer conditions are required, TA and TB are added to the substrate sequentially. Controls are prepared as described in Example 2. The amounts of TA and TB that are bound to each surface are determined by enzyme immunoassay.

Each substrate is evaluated to determine the ability of the combined surface bound TA and TB to inhibit complement activation when the substrate comes into contact with whole blood as described in Example 2.

### Example 15

### Immobilization of Complement Activation Regulator and Immunocapture Agent on Substrate with EGAP

In this example a substrate or device is coated with a regulator of complement activation and an immuno capture agent using EGAP. The purpose of the immunocapture agent is to remove unwanted components from the blood such as autoimmune antibodies, immunoglobulins, immune complexes, tumor antigens, or low-density lipoproteins.

In one variation, the immunocapture agent is immobilized with the regulator of complement activation as described in Example 5. In the other variation one part of the device is coated with EGAP/immunocapture agent and another part of the device is coated with EGAP/regulator of complement activation. In the later variation, the device is coated with EGAP as described in Example 2. The first selected region of the device is then incubated with a solution containing the immunocapture agent by either dip coating or controlled addition of the protein solution to a contained region of the device. The second selected region is then treated similarly with a solution containing the regulator of complement activation.

### Example 16

### Coating of Therapeutic Entities and Unmodified F108 on Substrate

In this example the device is coated in one region with one or more therapeutic entities as described in any one of the previous examples. The remainder of the device is coated with unmodified F108.

### Example 17

### Producing a Gel with EGAP

Sodium alginate was purified by extraction with 65% methanol. The purified sodium alginate was dissolved in water to produce a range of solution concentrations (0.3%-15% w/v). This solution was lightly crosslinked by adding calcium chloride (0.02% to 0.1%). PLURONIC F127, PLURONIC F68, EGAP, or a combination of these was added to chilled alginate gels at concentrations ranging from 10% to 25%. In some cases, gels were washed to remove excess calcium and subsequently dried by lyophilization. In other cases gels were formed without calcium initially, lyophilized and then calcium was added to the lyophilized structure. At approximately 15 to 20 wt %, PLURONIC F127 and EGAP will begin to form gels around room temperature. PLURONIC F68 has a lower critical solution temperature (LCST) above 40° C. By combining PLURONIC F127 and F68, it is possible to control the LCST of the mixture and produce a product that will flow below body temperature and will gel at body temperature. Formulations that gelled at room temperature and formulations gelled above body temperature were also produced. Formulations containing alginate and PLURONIC F127, PLURONIC F68, EGAP, or a combination of these, were also produced without calcium that displayed the ability to flow at room temperature. These were then injected into a water bath held at 37° C. and simultaneously mixed with calcium to form a gel at body temperature.

### Example 18

### Producing a Gel with a Therapeutic Entity

EGAP modified with a protease susceptible linker (EGAP-PSL) is produced as described in Example 1A or 2A. The EGAP-PSL is reacted with a therapeutic entity to produce a bioactive copolymer by incubating the EGAP-PSL with a mixture of EDC and N-hydroxysuccinimide in 4-morpholinoethanesulfonic acid (MES) for 15 minutes at room temperature to convert the carboxy terminus of the peptide to an amine reactive group. The EGAP-PSL is then incubated with a RCA protein or peptide of interest dissolved in MES for two hours at room temperature to produce an EGAP-RCA.

A gel having a therapeutic entity is then produced as described in Example 16 by substituting the EGAP-RCA for EGAP.

### Example 19

### Producing a Gel with Two or More Therapeutic Entities

EGAP-RCA is produced as described in Example 17. A second bioactive copolymer is produced by reacting EGAP-PSL produced as described in Example 1A or 2A with an antimicrobial peptide as described in Example 18 to produce an EGAP-AP. A gel having two different types of therapeutic entities is then produced as described in Example 16 by combining EGAP-RCA with EGAP-AP in a desired ratio and then substituting this mixture for EGAP.

### Example 20 (A)

### Producing a Gel with a Therapeutic Entity and Crosslinkable EGAP

EGAP having a terminal diacrylate group (EGAP-DA) is produced by dissolving PLURONIC F127 in a minimum amount of benzene under nitrogen atmosphere and then adding dichloromethane to obtain a 1:1 ratio of dichloromethane to benzene. The solution is then slowly cooled to 4° C. using an ice bath. Once the solution has cooled, 48 ul of triethylamine per gram of F127 is added. Finally, 26 ul Acryloyl chloride per gram of F127 is diluted in dichloromethane and added to the mixture slowly. The mixture is stirred overnight and the precipitated triethylammonium chloride is removed by filtration. The recovered solution is precipitated three times in cold diethyl ether. Conversion efficiency is determined by NMR analysis. EGAP-RCA is produced as described in Example 17. EGAP-DA is combined in water or buffer with an EGAP-RCA in the desired ratio to yield a total EGAP concentration of between 0.1% to 30%. The product produced is treated with irradiation to cause crosslinks to form between EGAP-DA entities (UV, gamma or e-beam).

### Example 20 (B)

### Producing Gel with a Therapeutic Entity and Crosslinkable EGAP

EGAP having an antimicrobial agent attached through a labile linker (EGAP-AB) is produced as described in Example 19. EGAP-DA is produced as described in Example 20(A). EGAP-DA is combined in water or buffer with EGAP-AB in the desired ratio to yield a total EGAP concentration of between 0.1% and 30%. In one case the product produced is treated with irradiation to cause crosslinks to form between EGAP-DA entities (UV, gamma or e-beam). In another case, the product is treated with heat to cause crosslinks to form between EGAP-DA entities. The gel may be used or stored in a hydrated state or may be freeze dried.

### Example 21

### Demonstrate Therapeutic Gel Adheres to Wound Bed

A porcine skin wound model is used to evaluate the ability of dressings to adhere to a wound bed. A gel is produced as described under Example 18 and freeze dried to produce the desired shape. A 17 mm diameter dressing sample, either dry or hydrated for predetermined time interval is be attached to a sample holder which is fixed to the load cell of a tensile tester. Porcine skin tissue purchased from a local grocery store is treated with dilute NaOH for 1 h before cutting into a disk shape, with a 17 mm diameter. The fat on the skin is removed with a scalpel. The porcine tissue, representing the wound bed, is fixed on the base of the tensile tester facing the dressing. The dressing is applied to the tissue and a preload is applied to the sample. This preload value is fixed based on the minimum force required to ensure an even contact between the two surfaces (dressing and porcine tissue). The load is applied for 3 min. Once the load is removed the tensile tester is initiated to move up with a constant speed of 4 mm/min up to the point of complete separation of the two surfaces. Both displacement and force of detachment (measured with the load cell) are recorded simultaneously and logged by the computer. Force versus displacement curves are analyzed subsequently in order to obtain the maximum force of detachment and to calculate the work of adhesion from the area beneath the curve (using the trapezoidal rule).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

The references listed below, as well as any other patents or publications referenced elsewhere herein, are all hereby incorporated by reference in their entireties.

### REFERENCES

1. Lee, J. H., Kopecek, J., and Andrade, J. D. (1989). Protein-resistant surfaces prepared by PEO-containing block copolymer surfactants. J Biomed Mater Res 23, 351-368.
2. Li, J. T., and Caldwell, K. D. (1996). Plasma protein interactions with Pluronic™-treated colloids. Colloids and Surfaces B: Biointerfaces 7, 9-22.
3. Li, J. T., Carlsson, J., Huang, S.-C., and Caldwell, K. D. (1996). Adsorption of poly(ethylene oxide)-containing block copolymers: a route to protein resistance. In Hydrophillic Polymers. Performance with environmental acceptability, J. E. Glass, ed. (Washington, D.C.: American Chemical Society), pp. 61-78.
4. McPherson, T., Park, K., and Jo, S. (2000). Grafting of biocompatible hydrophilic polymers onto inorganic and metal surfaces. In USPTO, United States Surgical (Norwalk, Conn.): USA.
5. Maechling-Strasser, C., Dejardin, P., Galin, J. C., Schmitt, A., Housse-Ferrari, V., Sebille, B., Mulvihill, J. N., and Cazenave, J. P. (1989). Synthesis and adsorption of a poly(N-acetylethyleneimine)-polyethyleneoxide-poly(N-acetylethyleneimine) triblock-copolymer at a silica/solution interface. Influence of its preadsorption on platelet adhesion and fibrinogen adsorption. J Biomed Mater Res 23, 1395-1410.
6. Winblade, N. D., Nikolic, I. D., Hoffman, A. S., and Hubbell, J. A. (2000). Blocking adhesion to cell and tissue surfaces by the chemisorption of a poly-L-lysine-graft-(poly(ethylene glycol); phenylboronic acid) copolymer. Biomacromolecules 1, 523-533.
7. Winblade, N. D., Schmokel, H., Baumann, M., Hoffman, A. S., and Hubbell, J. A. (2002). Sterically blocking adhesion of cells to biological surfaces with a surface-active copolymer containing poly(ethylene glycol) and phenylboronic acid. J Biomed Mater Res 59, 618-631.
8. Stewart, R. J., Caldwell, K. D., Ho, C. H., and Limberis, L. (2000). Metal-chelating surfactant. In USPTO, University of Utah: USA.
9. Caldwell, K., and Neff, J. A. (2002). End group activated polymers with oligonucleotide ligands. In World Intellectual Property Organization, allvivo, Inc.
10. Axen, R., and Carlsson, J. (1987). Therapeutically active compound and pharmaceutical composition containing the same. In USPTO, Pharmacia Aktiebolag: USA.
11. Carlsson, J., Axen, R., Drevin, H., and Lindgren, G. (1979). Pyridine disulfide compounds. In USPTO, Pharmacia Fine Chemicals AB: USA.
12. Basinska, T., and Caldwell, K. D. (1999). Colloid particles as immunodiagnostics: preparation and FFF characterization. In In Chromatography of Polymers: Hyphenated and Multidimensional Techniques., vol. 731. pp. 163-177, American Chemical Society Washington D. C.
13. Neff, J. A., Caldwell, K. D., and Tresco, P. A. (1998). A novel method for surface modification to promote cell attachment to hydrophobic substrates. J Biomed Mater Res 40, 511-519.
14. Neff, J. A., Tresco, P. A., and Caldwell, K. D. (1999). Surface modification for controlled studies of cell-ligand interactions. Biomaterials 20, 2377-2393.
15. Webb, K., Caldwell, K., and Tresco, P. A. (2000). Fibronectin immobilized by a novel surface treatment regulates fibroblast attachment and spreading. Crit. Rev Biomed Eng 28, 203-208.
16. Li, J. T., Carlsson, J., Lin, J. N., and Caldwell, K. D. (1996). Chemical modification of surface active poly(ethylene oxide)-poly(propylene oxide) triblock copolymers. Bioconjug Chem 7, 592-599.
17. Ripoche, J., Day, A. J., Harris, T. J., and Sim, R. B. (1988). The complete amino acid sequence of human complement factor H. Biochem J 249, 593-602.
18. Shim, W. S., Yoo, J. S., Bae, Y. H., and Lee, D. S. (2005) Novel injectable pH and temperature sensitive block copolymer hydrogel. Biomacromolecules 6, 2930-4.
19. He, C., Kim, S. W., and Lee, D. S. (2008) In situ gelling stimuli-sensitive block copolymer hydrogels for drug delivery. J Control Release 127, 189-207.

1. A bioactive agent delivery vehicle in the form of a matrix comprising a bioactive compound with the formula: 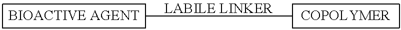
wherein the copolymer comprises one or more hydrophilic domains and at least one hydrophobic domain, the labile linker is a linkage that can be selectively cleaved to separate the bioactive agent from the copolymer upon exposure to a targeted physiologic stimulus that is produced in the in vivo environment in which the bioactive agent delivery vehicle is placed, and the bioactive agent is an agent that has a therapeutic effect.

2. The bioactive agent delivery vehicle according to claim 1, wherein the matrix is a liquid, gel, or foam.

3. The bioactive agent delivery vehicle according to claim 1, wherein the targeted physiologic stimulus is selected from the group consisting of hydrolysis, radiation, ultrasound, enzymatic, ionic, diffusion, barrier-mediated diffusion, competitive displacement, and liposomal disruption

4. The bioactive agent delivery vehicle according to claim 1, wherein the labile linker comprises a hydrolysable linker.

5. The bioactive agent delivery vehicle according to claim 1, wherein the labile linker comprises a protease susceptible labile linker.

6. The bioactive agent delivery vehicle according to claim 5, wherein the labile linker comprises a protease cleavage site and amino acid residues flanking the cleavage site that are varied to control the cleavage susceptibility of the cleavage site to the in vivo environment in which the bioactive agent delivery vehicle is placed, thereby providing control of the rate at which the bioactive agent is released from the bioactive agent delivery vehicle.

7. The bioactive agent delivery vehicle according to claim 1, wherein the bioactive agent is a pharmaceutical agent, protein, protein fragment, peptide, oligonucleotide, carbohydrate, proteoglycan, or antibody.

8. The bioactive agent delivery vehicle according to claim 1, wherein the hydrophilic domain comprises polyethylene oxide (PEO).

9. The bioactive agent delivery vehicle according to claim 1, wherein the hydrophobic domain comprises a polymer unit selected from the group consisting of polypropylene oxide (PPO), polybutadiene, poly(N-acetylethyleneimine), phenyl boronic acid, polyurethane, polymethylmethacrylate, poly(2-hydroxyethyl methacrylate), poly(n-butyl methacrylate) and poly(2-dimethylaminoethyl methacrylate), poly(n-butyl methacrylate) and polydimethyl sulfoxide and poly(carboxybetaine), polycaprolactone, poly(3-hydroxybutyrate, polystyrene, poly(butyl acrylate), poly(benzyloxytrimethylene carbonate), poly(-hexylthiophene), poly(ε-caprolactone), poly(2-vinylpyridine), poly(nitrobenzyl methacrylate), poly(t-butyl acrylate), poly(t-butyl methacrylate), poly(4-vinyl pyridine), poly(isobutylene), and poly(dimethylsiloxane).

10. The bioactive agent delivery vehicle according to claim 1, wherein the copolymer comprises polymer units selected from the group consisting of poly(amidoamine) and poly(propylene oxide) (PPO), poly(carboxybetaine) and poly(lactic-co-glycolic acid) (PLGA), Stearyl-poly-N-vinylpyrrolidone and dextran-polycaprolactone, poly(ethyl ethylene phosphate) and poly(-hydroxybutyrate), poly(acrylic acid) and polystyrene, polyisoprene and polystyrene, poly(L-amino acid) and poly(ester), poly(2-hydroxyethyl methacrylate) and poly(styrene), poly(n-butyl methacrylate) and poly(2-dimethylaminoethyl methacrylate), poly(n-butyl methacrylate) and poly(2-hydroxyethyl methacrylate), poly(vinyl alcohol) and poly(butyl acrylate), poly(styrene) and poly(acrylic acid), poly(acrylic acid) and poly(1-vinylpyrrolidone), poly(methacrylic acid) and poly(styrene), poly(vinyl pyrrolidone) and poly(benzyloxytrimethylene carbonate), poly(3-hexylthiophene) and poly(2-ethyl-2-oxazoline), poly(propylene oxide) and PEO, poly(butadiene) and PEO, PEO and poly(N-acetylethyleneimine), PEO and phenyl boronic acid, PEO and polyurethane, PEO and polymethylmethacrylate, and PEO and polydimethyl sulfoxide, poly(N,N-dimethylaminoethylmethacrylate) and PEO, poly(ε-caprolactone) and PEO, poly(lactic-co-glycolic acid) and PEO, N-isopropylacrylamide and PEO, polyL-lactide and PEO, poly(lysine) and PEO, poly(2-vinylpyridine) and PEO, poly(2-(diethylamino)ethyl methacrylate) and PEO, poly(isoprene) and PEO, poly(nitrobenzyl methacrylate) and PEO, poly(t-butyl acrylate) and PEO, poly(t-butyl methacrylate) and PEO, poly(2-hydroxyethyl methacrylate) and PEO, poly(4-vinyl pyridine) and PEO, poly(isobutylene) and PEO, poly(dimethylsiloxane) and PEO.

11. The bioactive agent delivery vehicle according to claim 1, further comprising a polymeric compound selected to modify physical properties or stability of the liquid, gel, foam, or matrix.

12. The bioactive agent delivery vehicle according to claim 11, wherein the polymeric compound is capable of forming a crosslinked network.

13. The bioactive agent delivery vehicle according to claim 11, wherein the polymeric compound is a polysaccharide.

14. A method of forming a medical device in the form of a gel matrix comprising:
obtaining a bioactive compound with the formula: 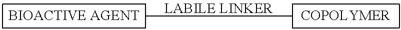
wherein the bioactive compound has a concentration in the range of about 10% to 30% by weight in an aqueous solvent, wherein the copolymer comprises one or more hydrophilic domains and at least one hydrophobic domain, the labile linker is a linkage that is selectively cleaved to separate the bioactive agent from the copolymer upon exposure to a targeted physiologic stimulus that is compatible with an in vivo environment in which the medical device is placed, and the bioactive agent is an agent that has a therapeutic effect; and
placing the bioactive compound solution at or near body temperature to form a gel.

15. The method according to claim 14, further comprising adding a polymeric compound to the bioactive compound to form a crosslinked network.

16. The method according to claim 15, wherein the polymeric compound is a polysaccharide.

17. The method according to claim 15, further comprising lyophilizing the bioactive compound.

18. A method of delivering a bioactive compound to an in vivo environment in a mammal comprising:
administering to the mammal a medical device in the form of a matrix comprising a bioactive compound with the formula: 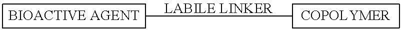
wherein the bioactive compound has a concentration in the range of about 10% to 30% by weight in an aqueous solvent, wherein the copolymer comprises one or more hydrophilic domains and at least one hydrophobic domain, the labile linker is a linkage that can be selectively cleaved to separate the bioactive agent from the copolymer upon exposure to a targeted physiologic stimulus, and the bioactive agent is an agent that has a therapeutic effect; and
cleaving the labile linker by exposure to a targeted physiologic stimulus in the mammal; thereby delivering the bioactive agent.

19. The method of claim 18, wherein the targeted physiologic stimulus is selected from the group consisting of hydrolysis, radiation, ultrasound, enzymatic, ionic, diffusion, barrier-mediated diffusion, competitive displacement, and liposomal disruption.

20. The method of claim 18, wherein the medical device is administered to the mammal by injecting a liquid matrix to the mammal and wherein the liquid matrix forms a gel at or near body temperature.

21. The method of claim 18, wherein the medical device is administered to the mammal to treat wounds.

22. The method of claim 18, wherein the medical device is administered to the mammal to inhibit post surgical adhesions and improve post surgical healing.
